(19) United States
(12) Patent Application Publication
(10) Pub. No.: US 2019/0125836 A1
(43) Pub. Date: May 2, 2019

(54) USE OF aFGF FOR PREVENTING OR TREATING DISEASES RELATED TO MUSCLE WASTING

(71) Applicant: Eusol Biotech Co., Ltd., Taipei (TW)

(72) Inventors: Hung-Kai CHEN, Taipei City (TW); Jing-Yi HUANG, Taipei City (TW); Huey-Wen HSIAO, Taipei City (TW); Che-Ming YEH, Kaohsiung City (TW)

(73) Assignee: Eusol Biotech Co., Ltd., Taipei (TW)

(21) Appl. No.: 16/175,420

(22) Filed: Oct. 30, 2018

Related U.S. Application Data
(60) Provisional application No. 62/579,379, filed on Oct. 31, 2017.

(51) Int. Cl. A61K 38/18; A61P 21/00; A61K 9/00

(52) U.S. Cl. CPC A61K 38/1825; A61K 9/0014; A61K 9/0019; A61P 21/00

(57) ABSTRACT

Disclosed are methods and compositions useful in preventing or treating a disease related to muscle wasting by using acidic fibroblast growth factor (aFGF).

### BACKGROUND OF THE INVENTION

Muscle wasting is recognized as being the consequence of aging, diseases, disorders or trauma, and affects millions of people (especially the elderly) worldwide. Specifically, acute muscle wasting is typically observed in patients during recovery of trauma or surgery; chronic muscle wasting is typically observed in the elderly people, or patients in severe diseases such as cancer, acquired immune deficiency (AIDS), chronic obstructive pulmonary disease (COPD), diabetes mellitus and heart failure. It is closely associated with poor quality of life as well as significantly increased morbidity and mortality. There are two commercially available steroids, nandrolone and oxandrolone, that are sometimes prescribed off-label for the treatment of cachexia in cancer patients.

Sarcopenia, which occurs with aging, is one of the diseases related to muscle wasting, and is characterized in the involuntary decline in muscle mass, strength and function. Sarcopenia increases the risk of loss of functional capacity in the elderly, which is not necessarily associated with other diseases. So far no drugs have been approved for sarcopenia, and various studies on the efficient control of sarcopenia have been conducted. It was found that the treatment with growth hormone (GH) can increase muscle mass. However, this treatment is very expensive and causes some undesired side effects such as shortening average life expectancy. As one of the most efficient methods for delaying the progression of sarcopenia, exercise with supplementary nutrition has been recommended, but it is very unsuitable for the elderly.

Muscular atrophy, which results from several conditions or disorders, is considered an emerging field for therapeutics. For example, stroke is one of the disorders that may cause long-term disability (e.g., hemiparesis or hemiplegia), leading to muscle abnormalities with a combination of denervation, disuse, remodeling and spasticity. In addition, given the stroke mortality rate keeps decreasing due to the improvement of stroke treatment in most developed countries, the proportion of patients transited to long-term disabled status increases significantly. However, less attention was paid on the pharmaceutical treatment of patients with post-stroke muscular atrophy.

A number of companies are developing drug candidates for muscle wasting diseases, including: Eli Lilly & Co., which has completed Phase 1 clinical studies and Phase 2 clinical trials for LY2495655, and Pfizer Inc., which is conducting Phase 2 clinical studies for PF-06252616, both of which are myostatin antibodies, to evaluate their ability to increase muscle mass and to improve muscle function in various patient populations (such as muscular atrophy and Duchenne Muscular Dystrophy); Novartis Corporation, which has conducted Phase 2/3 clinical trials for bimagrumab (BYM338), an ActR2B antibody, to evaluate its ability to build muscle in patients with various muscle-wasting conditions (such as sporadic inclusion body myositis, COPD with cachexia, and sarcopenia); Regeneron Pharmaceuticals, Inc., which has completed Phase 2 clinical trials for REGN1033, a myostatin antibody, in collaboration with Sanofi-Aventis for sarcopenia; Acceleron Pharma, which is developing ACE-083, a modified cysteine knot ligand trap of the TGF-β superfamily, for diseases in which improved muscle strength may provide a clinical benefit, such as inclusion body myositis and certain forms of muscular dystrophy; and GTx, Inc., which is developing ostarine, a selective androgen receptor modulator for cachexia. Ataluren (Translarna™), which was developed by PTC Therapeutics, is approved to treat people with Duchenne Muscular Dystrophy (DMD) in Europe, but with some restrictions (patients who have a nonsense mutation in the dystrophin gene, can walk, and are more than 5 years old). In addition, the U.S. Food and Drug Administration (FDA) declined to accept PTC Therapeutics' new drug application for ataluren, which was based on a clinical trial in which ataluren missed its primary endpoint. Another drug for DMD, Eteplirsen (EXONDYS 51™), was approved by the U.S. FDA in Sep. 2016, but only targets mutations in a region implicated in 13% of DMD cases.

Therefore, there is still a need to develop a drug and technology for preventing or treating diseases related to muscle wasting.

### SUMMARY OF THE INVENTION

The invention unexpectedly discovers that acid fibroblast growth factor (aFGF) is effective in treating diseases related to muscle wasting. When administering aFGF to a subject suffering from muscle wasting diseases, functional recovery of the degenerative or injured muscle was observed.

Accordingly, the present invention is directed to compositions for preventing or treating a disease related to muscle wasting, wherein the compositions comprise acidic fibroblast growth factor (aFGF). Also provided herein are methods for preventing or treating diseases related to muscle wasting comprising administrating an effective amount of aFGF.

It is to be understood that both the foregoing general description and the following detailed description are exemplary and explanatory only and are not restrictive of the invention.

### BRIEF DESCRIPTION OF THE DRAWINGS

FIG. 1 shows sciatic functional index (SFI) in sham, vehicle and ES135 treatment groups in rat sciatic nerve crush injury model.

FIG. 2 shows the grip strength in sham, vehicle and ES135 treatment groups in rat sciatic nerve crush injury model.

FIG. 3 shows the ratio of grip strength to body weight in sham, vehicle and ES135 treatment groups in rat sciatic nerve crush injury model.

FIG. 4 shows the gastrocnemius muscle weight and the ratio of gastrocnemius muscle weight to body weight (injured side) in sham, vehicle and ES135 treatment groups in rat sciatic nerve crush injury model.

FIG. 5 shows the gastrocnemius muscle weight and the ratio of gastrocnemius muscle weight to body weight (intact side) in sham, vehicle and ES135 treatment groups in rat sciatic nerve crush injury model.

FIG. 6 shows the soleus muscle weight and the ratio of soleus muscle weight to body weight (injured side) in sham, vehicle and ES135 treatment groups in rat sciatic nerve crush injury model.

FIG. 7 shows the soleus muscle weight and the ratio of soleus muscle weight to body weight (intact side) in sham, vehicle and ES135 treatment groups in rat sciatic nerve crush injury model.

FIG. 8 shows sciatic functional index (SFI) in sham, vehicle and ES135 treatment groups (with 5 different doses) in rat sciatic nerve crush injury model.

FIG. 9 shows the grip strength in sham, vehicle and ES135 treatment groups (with 5 different doses) in rat sciatic nerve crush injury model.

FIG. 10 shows the ratio of grip strength to body weight in sham, vehicle and ES135 treatment groups (with 5 different doses) in rat sciatic nerve crush injury model.

### DETAILED DESCRIPTION OF THE INVENTION

### I. Introduction

Provided herein are methods and compositions useful in preventing or treating a disease related to muscle wasting by using acidic fibroblast growth factor (aFGF). Based on the animal studies described herein, the inventors have shown that aFGF can increase muscle mass and strength as compared to the vehicle group.

### II. Definitions

The following abbreviations are used herein:

aFGF: acidic fibroblast growth factor
IM (i.m.): intramuscular injection
IV (i.v.): intravenous injection
IP (i.p.): intraperitoneal injection
SC (s.c.): subcutaneous injection
PO (p.o.): oral administration
Bid: bis in die (twice daily)

Unless defined otherwise, technical and scientific terms used herein have the same meaning as commonly understood by a person of ordinary skill in the art. Any methods, devices and materials similar or equivalent to those described herein can be used in the practice of this invention. The following definitions are provided to facilitate understanding of certain terms used frequently herein and are not meant to limit the scope of the present disclosure.

As used herein, the article “a” or “an” means one or more than one (that is, at least one) of the grammatical object of the article, unless otherwise made clear in the specific use of the article in only a singular sense.

As used herein, the term “muscle wasting” refers to a weakening, shrinking, and loss of muscle caused by aging, diseases (genetic or non-genetic), infection, injury or lack of use. Muscle wasting decreases a subject's strength and the ability to move.

As used herein, the term “aFGF” refers to a naturally-occurring, isolated, recombinant, or synthetically-produced aFGF which includes allelic variants, species homologs, or any modified peptide thereof. The modified peptide may be obtained such as by one or more deletions, insertions, substitutions or combinations thereof in the aFGF as defined above. In one embodiment of the invention, the modified aFGF is a peptide comprising a native human aFGF (154 amino acids in length) shortened by a deletion of 20 amino acids from N-terminal, and an addition of alanine before the shortened native human aFGF. For example, the modified aFGF may be a peptide consisting of the amino acid sequence of SEQ ID NO:1 (also called ES135), as described in U.S. application Ser. No. 12/482,041, the content of which is hereby incorporated by reference herein in its entirety. The amino acid sequence of SEQ ID NO:1 is as follows: Ala Asn Tyr Lys Lys Pro Lys Leu Leu Tyr Cys Ser Asn Gly Gly His Phe Leu Arg Ile Leu Pro Asp Gly Thr Val Asp Gly Thr Arg Asp Arg Ser Asp Gln His Ile Gln Leu Gln Leu Ser Ala Glu Ser Val Gly Glu Val Tyr Ile Lys Ser Thr Glu Thr Gly Gln Tyr Leu Ala Met Asp Thr Asp Gly Leu Leu Tyr Gly Ser Gln Thr Pro Asn Glu Glu Cys Leu Phe Leu Glu Arg Leu Glu Glu Asn His Tyr Asn Thr Tyr Ile Ser Lys Lys His Ala Glu Lys Asn Trp Phe Val Gly Leu Lys Lys Asn Gly Ser Cys Lys Arg Gly Pro Arg Thr His Tyr Gly Gln Lys Ala Ile Leu Phe Leu Pro Leu Pro Val Ser Ser Asp. In some embodiments, the sequence of a modified aFGF is at least 70%, 75%, 80%, 85%, 90%, 95%, 98%, or 100% identical to the amino acid sequence of SEQ ID NO:1. In some embodiments, the amino acid sequence of SEQ NO:1 has one or more modifications. For example, the amino acid sequence of SEQ NO:1 has an N-terminal phosphogluconoylation or gluconoylation as disclosed in U.S. application Ser. No. 14/508,118, the content of which is hereby incorporated by reference herein in its entirety.

As used herein, the term “prevent” or “preventing” refers to precluding, averting, obviating, forestalling, stopping, or hindering something from happening, especially by advance action.

As used herein, the term “treat” or “treating” refers to both therapeutic treatment and prophylactic or preventative measures, wherein the object is to prevent or slow down (lessen) an undesired physiological condition, disorder or disease, or to obtain beneficial or desired clinical results. For the purposes of this invention, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment.

As used herein, the term “therapeutically effective amount” or “effective amount” refers to an amount of aFGF to be used in the present invention which prevents, ameliorates or treats the symptoms accompanying a disease or condition referred to in this specification, or provides a desirable therapeutic effect. Therapeutic efficacy and toxicity of aFGF can be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50.

As used herein, the term “administering” when used in conjunction with a therapeutic means to administer a therapeutic directly into or onto a target tissue or to administer a therapeutic to a patient whereby the therapeutic positively impacts the tissue to which it is targeted. Thus, as used herein, the term “administering”, when used in conjunction with a compound, peptide or protein, can include, but is not limited to, providing a therapeutic into or onto the target tissue; providing a therapeutic systemically to a subject by, e.g., intravenous injection whereby the therapeutic reaches the target tissue. “Administering” a composition may be accomplished by oral, injection, topical administration, or by either method in combination with other known techniques.

The term “animal”, “subject” or “patient” as used herein includes, but is not limited to, humans and non-human vertebrates such as wild, domestic and farm animals, preferably humans.

As used herein, the term “motor function” refers to the biological activities of the tissues that affect or produce movement in an animal. Such tissues include, without limitation, muscles and motor neurons.

As used herein, the term “muscle strength” refers to the amount of force a muscle, or muscle groups in sum, can exert. Muscle strength may be evaluated by a variety of methods such as grip strength test, one repetition maximum strength test, time-dependent tests of muscle endurance, time-dependent tests of muscle fatigue, or time-dependent tests of muscle endurance and fatigue, and so forth.

As used herein, the term “muscle mass” refers to the amount or size of muscle or muscle groups, as expressed by muscle weight, mass, area, or volume. Muscle mass may also be expressed as total lean body mass, lean body mass of a body compartment such as the leg, or cross-sectional area of a leg or arm compartment. The volume or mass of the muscle can be determined using any known or otherwise effective technique that provides muscle area, volume or mass, such as dual energy X-ray absoptiometry (DEXA), or using visual or imaging techniques such as magnetic resonance imaging (MM) or computed tomography (CT) scans.

### III. Embodiments of the Invention

In one embodiment, the invention is directed to a pharmaceutical composition for preventing or treating a disease related to muscle wasting in a subject, comprising acidic fibroblast growth factor (aFGF). The disease includes but is not limited to cachexia as a result of, for example, rapid loss of muscle in all cancers; muscular atrophy resulting from several conditions or disorders, including but not limited to immobility, bedridden, infection (such as poliomyelitis), malnutrition, hormonal deregulation, fracture (broken bone), surgery (such as hip replacement), burns, sepsis, stroke, diabetes mellitus, chronic kidney disease, heart failure, chronic obstructive pulmonary disease (COPD), and acquired immune deficiency syndrome (AIDS); spinal muscular atrophy (SMA); muscular dystrophy (such as Becker muscular dystrophy, congenital muscular dystrophy, Duchenne muscular dystrophy, distal muscular dystrophy, Emery-Dreifuss muscular dystrophy, facioscapulohumeral muscular dystrophy, limb-girdle muscular dystrophy, myotonic muscular dystrophy, oculopharyngeal muscular dystrophy); sarcopenia (aging-related muscle wasting); myopenia; dynapenia; inclusion body myositis; and combinations thereof. Preferably, the pharmaceutical composition of the present invention can be used in the prevention or treatment of sarcopenia, myotonic muscular dystrophy, cachexia, muscular atrophy resulting from fracture, surgery, stroke or combinations thereof. More preferably, the pharmaceutical composition of the present invention can be used in the prevention or treatment of sarcopenia. Specifically, the term “sarcopenia” as used herein means the gradual decrease in skeletal muscle mass caused by aging, which can directly cause a decrease in muscle strength, resulting in a decrease and impairment in various physical functions.

In one aspect, the invention is directed to a method for preventing or treating a disease related to muscle wasting in a subject, comprising administering a therapeutically effective amount of acidic fibroblast growth factor (aFGF) to the subject. The therapeutic effects of aFGF have been proven in the animal model for muscle wasting as described herein.

In some embodiments, the animal model for muscle wasting is rat sciatic nerve crush injury model, which is designed to demonstrate the effects of an agent in promoting muscle growth and functional recovery. Preferably, the rat sciatic nerve crush injury model is prepared by clamping the left sciatic nerve for 60 seconds using pincers with 2 mm width.

In some embodiments, aFGF is administered in rat sciatic nerve crush injury model for the evaluation of its effect on muscle growth and functional recovery. Preferably, aFGF is administered intramuscularly (IM). In some embodiments, aFGF is administered once, twice, thrice per day or less, e.g., every second day, every third day, every week, every other week, or less. As demonstrated in the present invention, aFGF is administered intramuscularly twice daily for 21 days (bid×21). The detailed study procedures are shown in the Examples below.

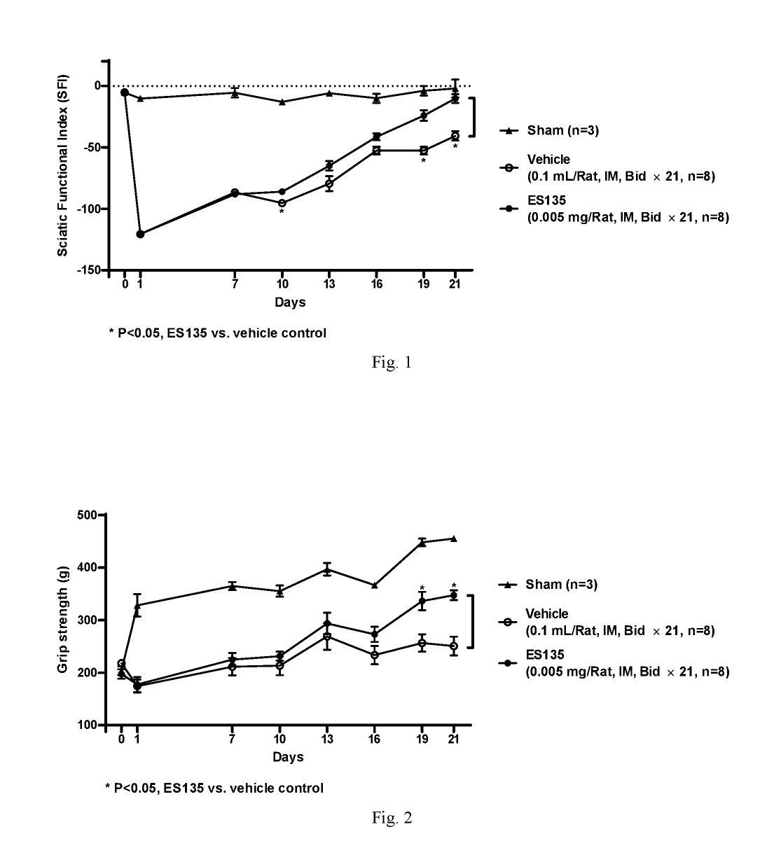

In some embodiments, administration of aFGF can improve motor function in a subject. Motor function can be determined by gait speed or sciatic functional index (SFI). As described herein, functional recovery of rat was evaluated by the SFI value after sciatic nerve injury. The SFI value in the vehicle control group was reduced by sciatic nerve crush and gradually returned to the pre-surgical baseline (FIG. 1). On the other hand, multiple administrations of modified aFGF (ES135) at 0.005 mg/animal improved SFI on Days 19 and 21 as compared to the vehicle control, suggesting a better functional recovery (P<0.05, one-way ANOVA with Dunnett's posthoc test; FIG. 1).

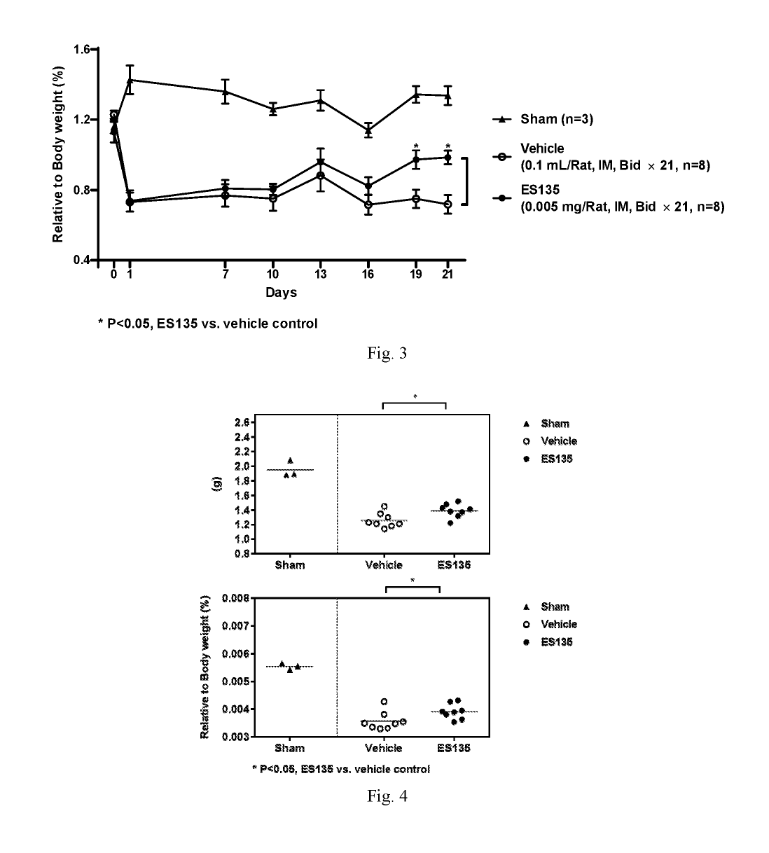

In some embodiments, administration of aFGF can improve muscle strength in a subject. Methods for determining muscle strength include, but are not limited to, grip strength test, one repetition maximum strength test, time-dependent tests of muscle endurance, time-dependent tests of muscle fatigue, or time-dependent tests of muscle endurance and fatigue. As described herein, muscle strength of rat was evaluated by grip strength after sciatic nerve injury. The grip strength and normalized grip strength (relative to body weight) in the vehicle control group were reduced by sciatic nerve crush and gradually returned to the pre-surgical baseline (FIGS. 2-3). Like SFI value, multiple administrations of ES135 improved grip strength on Days 19 and 21 as compared to the vehicle control, also suggesting a better functional recovery (P<0.05, one-way ANOVA with Dunnett's posthoc test; FIGS. 2-3).

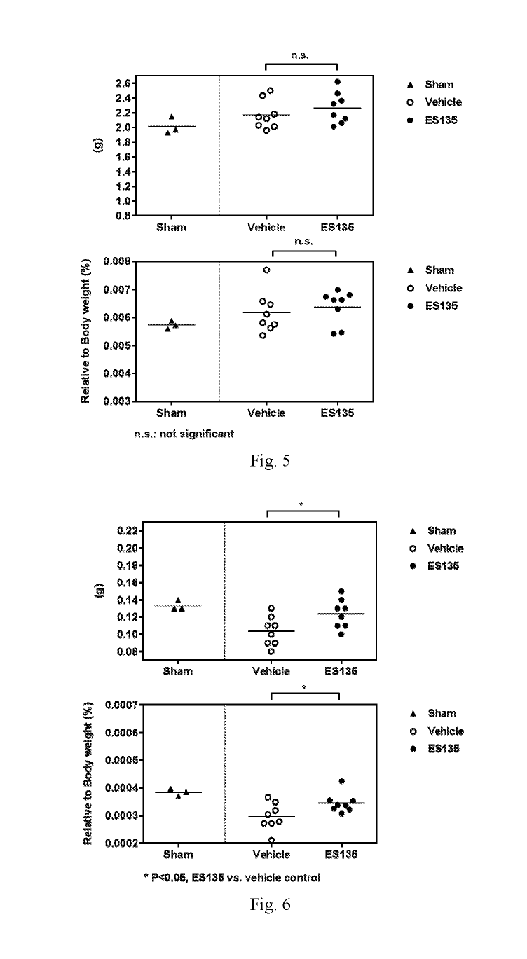

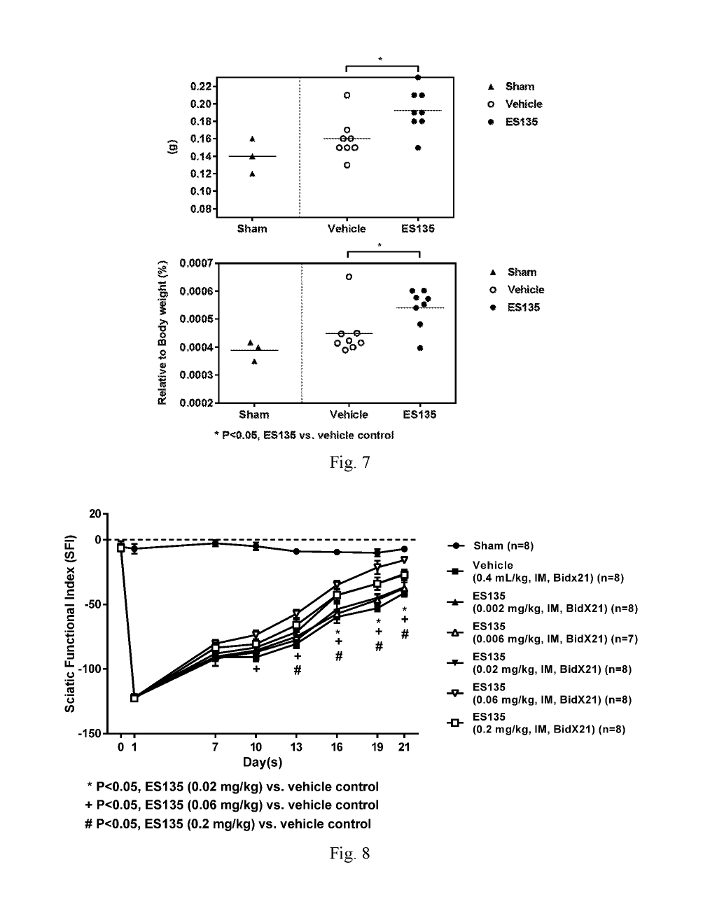

In some embodiments, administration of aFGF can increase muscle mass. Methods for measuring muscle mass include, but are not limited to, dual energy X-ray absorptiometry (DEXA), magnetic resonance imaging (MM) or computed tomography (CT) scans, invasive approach such as muscle harvest and weight by an electronic balance. As described herein, muscle mass of rat was measured by an electronic balance. Specifically, after the rats were sacrificed, gastrocnemius and soleus muscles were harvested and weighed from both injured and intact hind limbs, and the ratio of the gastrocnemius muscle weight and soleus muscle weight to body weight were also calculated (FIGS. 4-7). Compared to the sham control, gastrocnemius and soleus muscle weights in injured side were reduced by the nerve crush in vehicle control group, whereas ES135 treatment group attenuated the reduction of muscle mass (P<0.05, unpaired Student's t-test; FIGS. 4 and 6). In addition, the ES135 administration increased soleus muscle weight in intact side and its ratio (relative to body weight) to the vehicle control (P<0.05, unpaired Student's t-test; FIG. 7). Although not statistically significant, the ES135 administration slightly increased gastrocnemius muscle weight in intact side and its ratio (relative to body weight) to the vehicle control (P>0.05, unpaired Student's t-test; FIG. 5).

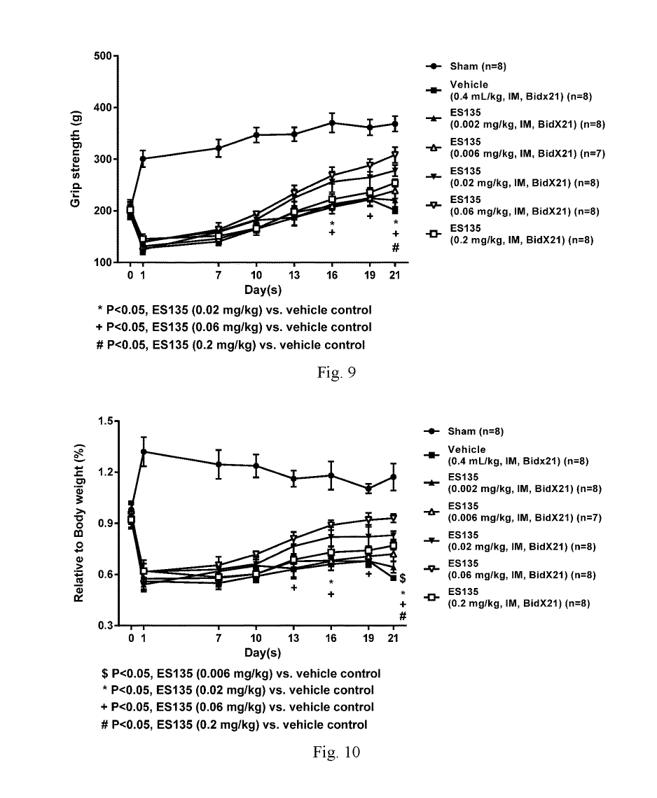

In some embodiments, the dose of aFGF administered is equivalent to 0.001-1 mg aFGF per kg body weight (i.e., 0.001-1 mg/kg) of the subject, e.g., equivalent to 0.001-0.01, 0.01-0.05, 0.05-0.1, 0.1-0.2, 0.1-0.4, 0.05, 0.1, 0.2, 0.3, 0.4, 0.5 or higher mg aFGF per kg body weight. In order to test whether the functional recovery by ES135 treatment is in a dose-dependent matter, we measured the SFI values and grip strengths in rat sciatic nerve crush injury model treated with different doses (from 0.002 to 0.2 mg/kg) of ES135, which were injected intramuscularly twice daily for 21 days (bid×21). The data indicated that ES135 improved the SFI values in 0.02, 0.06 and 0.2 mg/kg treatment groups from Day 10 to Day 21, as compared to the vehicle control group (P<0.05, one-way ANOVA with Dunnett's posthoc test; FIG. 8). As for grip strength and normalized grip strength (relative to body weight), the data showed that ES135 also improved the grip strengths in 0.006, 0.02, 0.06 and 0.2 mg/kg treatment groups from Day 13 to Day 21, as compared to vehicle control group (P<0.05, one-way ANOVA with Dunnett's posthoc test; FIGS. 9-10). Dose-dependent functional improvement in both assessments was observed, while the ES135 treatment group with the dose of 0.06 mg/kg (but not 0.2 mg/kg) showed the most significant improvement.

In conclusion, multiple treatments of ES135 significantly attenuated the sciatic nerve crush induced motor dysfunctions and increased muscle mass in injured hind limbs.

According to the invention, aFGF may be constituted into any form suitable for the mode of administration selected. Preferably, aFGF is administered subcutaneously, topically, intraneurally, intravenously or intramuscularly. According to the invention, a skilled person in the art may easily determine the administrative pathway and the dosage dependent on his experiments or the physiological status of the patients suffering from diseases related to muscle wasting, i.e. age, body weight, severity, or dosage. In an embodiment of the invention, aFGF is injected intramuscularly into biceps femoris muscle.

### EXAMPLES

The present invention is more specifically explained by the following examples. However, it should be noted that the present invention is not limited to these examples in any manner.

### Sciatic Nerve Crush Surgery and Administration of aFGF in Rats

Groups of 3 or 8 male SD rats weighing 250±20 g were employed. Under pentobarbital (50 mg/kg, 5 mL/kg, IP) anesthesia, the left sciatic nerve was exposed at mid-thigh level. Surgery was performed by clamping the left sciatic nerve for 60 seconds using pincers with 2 mm width; complete crush was confirmed by the presence of a translucent band across the nerve. The incision was then closed in layers (muscle and skin) with absorbable sutures. The sham group received sham surgery only (no sciatic nerve crush). One day after the surgery, the rats with sciatic nerve crush are randomized to vehicle and ES135 treatment groups based on the sciatic functional index (SFI). Vehicle and ES135 were injected intramuscularly (i.m.) twice daily for 21 days (bid×21).

### Sciatic Functional Index (SFI)

SFI was evaluated prior to and on Days 1 (pre-dose), 7, 10, 13, 16, 19, and 21 after surgery. The hind feet were pressed down onto a stamp pad soaked with water-soluble blue ink (face & body paint). Rats were immediately allowed to walk along a confined walkway (7.5 cm W, 60 cm L) with a dark shelter at the end of the corridor. The walkway was lined with paper on the floor to capture the footprints. The following measurements were taken from the footprints: (1) distance from the heel to the third toe, the print length (PL); (2) distance from the first to fifth toe, the toe spread (TS); and (3) distance from the second to the fourth toe, the intermediary toe spread (ITS).

All three measurements were taken from the experimental (E, undergoing sciatic nerve crush) and normal (N) limbs. Three factors that comprise the SFI were calculated as follows: (1) print length factor (PLF)=(EPL−NPL)/NPL; (2) toe spread factor (TSF)=(EST−NST)/NST; and (3) intermediary toe spread factor (ITF)=(EIT−NIT)/NIT. Using these data, the SFI, which indicated the differences between the injured and the intact contralateral paw, was calculated by the following formula.

SFI=−38.3[(EPL−NPL)/NPL]+109.5[(ETS−NTS)/NTS]+13.3[(EIT−NIT)/NIT]−8.8.

An SFI equal to −100 indicated significant impairment, whereas an SFI oscillating around 0 was considered to reflect normal function.

### Grip Strength Measurement

Hind limb grip strength was measured prior to and on Days 1 (pre-dose), 7, 10, 13, 16, 19, and 21 after surgery as tension force using a computerized grip strength meter (UGO Basile, Italy).

Muscle Harvest and Mass Ratio Calculation

Immediately after animals were sacrificed on Day 22, gastrocnemius and soleus muscles and fiber cross-sectional area (gastrocnemius) were dissected and carefully harvested from both intact and injured sides and weighed while still wet using an electronic balance. Values were expressed as a ratio of the wet weight of the gastrocnemius muscle or soleus muscle (injured or intact side) to whole body weight. Half of gastrocnemius muscles were snap-frozen and the other half was formalin-fixed for further histopathological analysis. Half of soleus muscles were snap-frozen, and the other half was kept in RNAlater. The weights of quadriceps muscles were recorded.

All data are expressed as means ±SEM. Unpaired Student's t-test were applied to assess statistical significance between vehicle control groups and ES135 treatment groups. One-way ANOVA with Dunnett's posthoc test was used to compare between the vehicle control groups and ES135 treatment groups. P<0.05 was considered statistically significant. Statistical analysis was performed using GraphPad Prism 7.0 software (GraphPad Software, Inc., USA).

While the foregoing written description of the invention enables one of ordinary skill in the art to make and use what is considered presently to be the best mode thereof, those of ordinary skill in the art will understand and appreciate the existence of variations, combinations, and equivalents of the specific embodiments, methods, and examples herein. The invention should therefore not be limited by the above described embodiments, methods, and examples, but by all embodiments and methods within the scope and spirit of the invention.

What is claimed is:

1. A pharmaceutical composition for preventing or treating a disease related to muscle wasting comprising acidic fibroblast growth factor (aFGF).

2. The pharmaceutical composition according to claim 1, wherein the aFGF has the amino acid sequence of SEQ ID NO: 1.

3. The pharmaceutical composition according to claim 2, wherein the aFGF consists of the amino acid sequence of SEQ ID NO: 1.

4. The pharmaceutical composition according to claim 1, wherein the disease is selected from the group consisting of sarcopenia, myotonic muscular dystrophy, cachexia, muscular atrophy resulting from fracture, surgery or stroke, and combinations thereof.

5. A method for preventing or treating a disease related to muscle wasting in a subject, comprising administering a therapeutically effective amount of acidic fibroblast growth factor (aFGF) to the subject.

6. The method according to claim 5, wherein the aFGF is administered subcutaneously, topically, intraneurally, intraperitoneally, intravenously or intramuscularly.

7. The method according to claim 6, wherein the aFGF is administered intramuscularly.

8. The method according to claim 5, wherein the aFGF has the amino acid sequence of SEQ ID NO: 1.

9. The method according to claim 8, wherein the aFGF consists of the amino acid sequence of SEQ ID NO: 1.

10. The method according to claim 5, wherein the disease is selected from the group consisting of sarcopenia, myotonic muscular dystrophy, cachexia, muscular atrophy resulting from fracture, surgery or stroke, and combinations thereof.

11. The method according to claim 5, wherein the aFGF is administered at a dose of 0.01 to 1 mg/kg once or twice a day.

12. A pharmaceutical composition for improving motor function or muscle strength comprising acidic fibroblast growth factor (aFGF).

13. A method for improving motor function or muscle strength in a subject, comprising administering an effective amount of acidic fibroblast growth factor (aFGF) to the subject.

14. A pharmaceutical composition for increasing muscle mass comprising acidic fibroblast growth factor (aFGF).

15. A method for increasing muscle mass in a subject, comprising administering an effective amount of acidic fibroblast growth factor (aFGF) to the subject.


SEQUENCE LISTING (1 sequence)

SEQ ID NO: 1 - protein, 135 aa, Artificial Sequence
  Peptide modified from Homo sapiens Acid-      Fibroblast Growth Factor (a-FGF)
ANYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD